(19) United States
(12) Patent Application Publication
(10) Pub. No.: US 2020/0000709 A1
(43) Pub. Date: Jan. 2, 2020

(54) COMBINATION OF BIOELECTRICAL STIMULATOR AND PLATELET-RICH FIBRIN FOR ACCELERATED HEALING AND REGENERATION

(71) Applicant: Cal-X Stars Business Accelerator, Inc., Playa Vista, CA (US)

(72) Inventors: Howard J. Leonhardt, Playa Vista, CA (US); Richard J. Miron, Venice, FL (US); Valerie Kanter, Los Angeles, CA (US)

(21) Appl. No.: 16/459,117

(22) Filed: Jul. 1, 2019

Related U.S. Application Data
(60) Provisional application No. 62/691,843, filed on Jun. 29, 2018.

(51) Int. Cl. A61K 9/00; A61K 38/36; A61K 38/18; A61K 35/19; A61K 35/15; B01D 21/26

(52) U.S. Cl. CPC A61K 9/0009; A61K 38/36; A61K 38/1866; A61K 38/1808; B01D 21/262; A61K 38/1858; A61K 35/19; A61K 35/15; A61K 38/1841

(57) ABSTRACT

Means and methods utilizing a combination of bioelectrical stimulator and platelet-rich fibrin for accelerated tissue or wound healing and regeneration is described. The system bioelectrically stimulates the centrifuge, test tube, and/or subject to produce enhanced levels of, e.g., SDF, PDGF, HGF, VEGF, IGF, Sonic hedgehog, klotho, and/or tropoelastin. The described system produces much higher levels of regenerative proteins delivered over an extended period of time.

### CROSS-REFERENCE TO RELATED APPLICATIONS

This application claims the benefit under 35 USC § 119 of U.S. Provisional Patent Application Ser. No. 62/691,843, filed Jun. 29, 2018, the contents of which are incorporated herein in its entirety by this reference.

### TECHNICAL FIELD

The application relates generally to the field of medical devices and associated treatments, and to precise bioelectrical stimulation of a subject's tissue, potentially augmented with the administration of a composition comprising, among other things, stem cells and nutrients, useful to stimulate and treat the subject, the subject's tissue(s), the subject's organ(s), and/or the subject's cells. More specifically described is a personalized bioelectric cancer tumor eradication therapy. Also described is a multi-modality bioelectric therapy protocol for cancer tumor treatment.

### BACKGROUND

Platelet-rich Fibrin (“PRF”) is a biological matrix derived from peripheral blood that forms a fibrin scaffold containing cells and growth factors. While the scaffolds promotes cellular recruitment and growth factor release into defect sites, reports from the literature suggest that the delivery of growth factors is not specific to tissue type.

As would be understood by one of ordinary skill in the art, PRF or leucocyte- and platelet-rich fibrin (“L-PRF”) is a second-generation Platelet Rich Plasma (“PRP”), where autologous platelets and leucocytes are present in a complex fibrin matrix that accelerate the healing of soft and hard tissues. PRF is used as a tissue-engineering scaffold for, e.g., endodontics.

To obtain PRF, a quantity of blood is drawn quickly from a subject into test tubes (without an anticoagulant) and then centrifuged immediately. Blood can be centrifuged using, e.g., a tabletop centrifuge for at least ten minutes at 3,000 revolution per minute (“rpm”). The resultant product has the following three layers: (a) topmost layer with platelet poor plasma, (b) PRF clot in the middle, and (c) red blood cells (“RBC”) at the bottom. PRF is available as a fibrin clot. PRF clot can be removed from the test tube using a sterile tweezer-like instrument. After lifting, the RBC layer attached to the PRF clot can be carefully removed using, e.g., a sterilized scissor.

Platelet activation in response to tissue damage occurs during the process of making PRF, releasing several biologically active proteins including: platelet alpha granules, platelet-derived growth factor (“PGDF”), transforming growth factors-β (“TGF-(3”), vascular endothelial growth factor (“VEGF”), and epidermal growth factor (“EGF”).

Upon activation, bioelectrical stimulation of tissues has been shown to sequentially and accurately delivery specific growth factors.

### SUMMARY

Described are means, methods, and systems for bio-electrically stimulating PRF before, during, and/or after entry into a subject's body to further stimulate more specific and robust tissue regeneration/healing.

For example, the described system can be used to bioelectrically stimulate the centrifuge, test tube, Petri dish, and/or subject to produce enhanced levels of, e.g., SDF-1, PDGF, HGF, VEGF, IGF, Sonic hedgehog, klotho, and/or tropoelastin. Much higher levels of regenerative proteins delivered over an extended period of time.

Described herein are means and methods that utilize a combination of bioelectrical stimulator and platelet-rich fibrin for accelerated tissue or wound healing and regeneration. The described system produces much higher levels of regenerative proteins delivered over an extended period of time.

Described is a method of accelerating tissue healing and regeneration in a subject, wherein the method includes administering platelet-rich fibrin (“PRF”) and/or leucocyte- and platelet-rich fibrin (“L-PRF”) to the subject, and bioelectrically stimulating the subject and/or the PRF and/or L-PRF to increase and extend PRF or L-PRF regenerative protein expression, so as to accelerate healing and regeneration in the subject. In such a method, the PRF or L-PRF regenerative protein is typically selected from the group consisting of platelet alpha granules, platelet-derived growth factor (“PGDF”), transforming growth factors-β (“TGF-β”), vascular endothelial growth factor (“VEGF”), and epidermal growth factor (“EGF”).

Extended Growth Factor Release or “ER-PRF” (e.g., BIO PRF® from Miron Research and Development in Dentistry LLC of Florida, US) is thought to release growth factors for from 21 to 28 days in contrast to 7 to 10 days for PRF.

Also described is a method of encouraging tissue healing and regeneration in a subject, wherein the improvement comprises using a combination of bioelectrical stimulation and utilization of platelet-rich fibrin (“PRF”) and/or leucocyte- and platelet-rich fibrin (“L-PRF”) to increase and extend PRF regenerative protein expression to enhance and/or accelerate tissue healing and regeneration in the subject. In such a method, the PRF or L-PRF regenerative protein is typically selected from the group consisting of platelet alpha granules, PGDF, TGF-β”, VEGF, and EGF.

Further described is a method of modifying a PRF centrifuge of the type having a chamber for containing the blood to form the PRF, the method comprising: providing leads for bioelectrical stimulation of the chamber during a centrifugation process. Also included is a centrifuge produced according to such a method.

The described process, system, and associated methods are particularly useful in skin and hair regeneration in a subject.

### DETAILED DESCRIPTION

As previously identified herein, platelet-rich fibrin is a biological matrix derived from peripheral blood that forms a “fibrin scaffold” containing cells and, e.g., the previously described growth factors. L-PRF may also be used herein, substituting for or augmenting the PRF. While the scaffold promotes cellular recruitment and growth factor release into defect sites, reports from the literature suggest that the delivery of growth factors is unspecific to the tissue type. Bioelectrical stimulation of tissues has been shown to sequentially and accurately delivery specific growth factors upon activation. Described is a method for stimulating PRF before, during, and/or after entry into the body to further stimulate more specific and robust tissue regeneration/healing.

The described precise bioelectrical signals increase the number of regenerative cells in PRF compositions and increase the volume and time of expression of regeneration promoting growth factors released both in vitro and in vivo.

Previous bioelectrical stimulators failed to have the precise control of stem cell homing, proliferation, and/or differentiation and controlled protein expressions. PRF therapies (whether delivered as a fibrin matrix or a liquid) failed to stimulate precise growth factor delivery for optimal healing and regeneration or various tissues.

This disclosure is an improvement over what currently exists. Previous PRF therapies (whether delivered as a fibrin matrix or a liquid) failed to stimulate precise growth factor delivery for optimal healing and regeneration or various tissues. The system of U.S. Patent Application Publication US 2018-0064935-A1 to Leonhardt et al. (Mar. 8, 2018), the contents of the entirety of which are incorporated herein by this reference, can be utilized to increase (e.g., local) stem cells and regenerative proteins in the subject.

The incorporated U.S. Patent Application Publication US 2018-0064935-A1 to Leonhardt et al. describes particular bioelectric signals and times useful to induce a mammalian cell to produce, for example, stromal cell-derived factor 1 (“SDF1”), insulin-like growth factor 1 (“IGF1”), hepatocyte growth factor (“HGF”), epidermal growth factor (“EGF”), platelet-derived growth factor (“PDGF”), vascular endothelial growth factor (“VEGF”), hypoxia-inducible factor 1-alpha (“HIF-1-alpha”), endothelial NOS (“eNOS”), activin A, activin B, interleukin 6 (“IL-6”), follistatin, tropoelastin, GDF-10, GDF-11, neurogenin 3, FGF, TGF, tumor necrosis factor alpha (“TNF A”), receptor activator of nuclear factor kappa-B ligand (“RANKL”), osteoprotegerin (“OPG”), and any combination thereof.

These bioelectric signals are applied to, for example, the centrifuge which is used to make the PRF, the dish or test tube in which the PRF is incubated, and/or the subject undergoing treatment to enhance, for example, the amount of SDF1, PDGF, HGF, VEGF, tropoelastin, klotho, sonic hedgehog, IGF, etc.

The described method and product produce much higher levels of regenerative proteins delivered over an extended period of time.

Also, described is a centrifuge (e.g., a BIO-PRF Centrifuge Machine from Nextgen Biomaterials) that has been modified to contain a built-in bioelectrical stimulator. The device can also allow for the PRF (either in membrane or liquid formulation) to be placed directly onto or inside it, with built-in sensors allowing for the transfer of the bioelectrical signals. For specific tissues, a bioelectrical organ reader is included as well as a bioelectrical signal program (zip or other) disk or cartridge that allows for a specific program for each specific regenerative protocol. Such a device could also include bioelectrical stimulation leads or probes as well as some conductive matrix to transfer the signals towards the PRF.

In certain embodiments, the system described herein includes:
1. Bioelectrical stimulator signal generator (see, e.g., the incorporated U.S. Patent Application Publication US 2018-0064935-A1 to Leonhardt).
2. Bioelectrical organ reader
3. PRF centrifuge (such as a Labnet Spectrafuge 6C Benchtop Centrifuge from Cole-Parmer of Vernon Hills, Ill., US, a PRF Centrifuge/GRF Kit by Dental USA, McHenry, Ill., US, or EZMINISPIN, CosmoFrance, Inc. of Miami, Fla., US)
4. PRF patient kit
5. Bioelectric signal program media (e.g., zip disk or cartridge)
6. WiFi-Based Use Reader
7. Bioelectrical stimulation leads and/or probes
8. Conductive matrix

In certain embodiments, an EZPRF Kit (available from CosmoFrance, Inc. of Miami, Fla., US) can be utilized with the system.

Relationship Between The Components:

The bioelectrical stimulator may be used in the PRF centrifuge externally, applied to the liquid PRF or PRF membrane, or applied to the patient with PRF.

How The Invention Works:

Bioelectrically stimulating positive and negative electrodes cross PRF composition to control stem cell and protein expression activity.

How To Make a System according to the disclosure:

In certain embodiments, the PRF membrane or PRF liquid is bio-electrically stimulated during the centrifugation process, e.g., either following the centrifugation process for the PRF or following injection of the PRF into the human body. For instance, 1) a bioelectrical stimulation device is installed within the periphery of a centrifuge and the bioelectrical signals are applied during the centrifugation process. 2) A PRF membrane is created through centrifugation, and then placed on a tray where a bioelectrical signal is passed through the PRF membrane or PRF liquid to stimulate upregulation of specific target genes. 3) The PRF could be implanted or injected in vivo and then PRF stimulation could be applied to the subject.

This technology typically utilizes a centrifuge and all its associated components as well as a bioelectrical stimulator and all its necessary components. The additional use of a bioelectrical stimulation board on the centrifuge to place PRF membranes or PRF liquid facilitating the transfer of bioelectrical stimulation would additionally make the system work better.

The devices can be applied in different sequences. For instance, a patient could first receive the PRF injections followed by bioelectrical stimulation, and/or the patient may receive the bioelectric stimulation first to prepare the tissue with the selected growth factors, followed by PRF injection/implantation.

How to Use the System:

In certain embodiments, a bioelectrical signals is applied directly to the PRF membrane. Thereafter, the highly stimulated PRF membrane releases higher levels of growth factors, which would then be utilized for a variety of regenerative procedures as outlined herein.

Additionally, the technology can be utilized for regenerating various tissue(s) in the subject's (e.g., human or other mammalian) body.

Since PRF promotes new blood flow, it facilitates regeneration of many tissues. Since bioelectrical stimulation can stimulate virtually any specific gene to be upregulated, it can also be utilized in most tissue(s). Furthermore, this technology can also be utilized in the veterinary field for the healing of defects in animals.

Also described herein is a centrifuge that contains a built in bioelectrical stimulator. It can also incorporate a device that would allow for the PRF (either in membrane or liquid formulation) to be placed directly onto or inside it, with built-in sensors that would allow the transfer of the bioelectric signals. For specific tissues, a bioelectrical organ reader could be utilized as well as a bioelectrical signal program computer readable media (e.g., zip disk or cartridge) that would allow for the specific program for each specific regenerative protocol. The device would also include bioelectric stimulation leads or probes as well as some conductive matrix to transfer the signals towards the PRF.

The invention is further described with the aid of the following illustrative Examples.

### EXAMPLE 1

A middle-aged Caucasian woman presented with thinning hair of the scalp. The number of hairs per square centimeter were measured. PRF together with the herein described bioelectric stimulation yields a 20-30% hair density increase.

### EXAMPLE 2

A post-menopausal Caucasian woman presented with thinning and damaged skin of the face. Microneedle abrasion, together with the herein described PRF and bioelectric stimulation yielded a noticeable improvement.

What is claimed is:

1. A method of accelerating tissue healing and tissue regeneration in a subject, the method comprising:
administering platelet-rich fibrin (“PRF”), Extended Growth Factor Release platelet-rich fibrin (“ER-PRF”), and/or leucocyte- and platelet-rich fibrin (“L-PRF”) to the subject, and
bioelectrically stimulating the subject and/or the PRF, ER-PRF, and/or L-PRF to increase and extend PRF or L-PRF regenerative protein expression,
so as to accelerate healing and tissue regeneration in the subject.

2. The method according to claim 1, wherein the PRF or L-PRF regenerative protein is selected from the group consisting of platelet alpha granules, platelet-derived growth factor (“PGDF”), transforming growth factors-β (“TGF-β”), vascular endothelial growth factor (“VEGF”), and epidermal growth factor (“EGF”).

3. In a method of encouraging tissue healing and regeneration in a subject, the improvement comprising:
using a combination of bioelectrical stimulation and utilization of platelet-rich fibrin (“PRF”), Extended Growth Factor Release platelet-rich fibrin (“ER-PRF”), and/or leucocyte- and platelet-rich fibrin (“L-PRF”) to increase and extend PRF regenerative protein expression to enhance and/or accelerate tissue healing and regeneration in the subject.

4. The method according to claim 3, wherein the PRF, ER-PRF, and/or L-PRF regenerative protein is selected from the group consisting of platelet alpha granules, platelet-derived growth factor (“PGDF”), transforming growth factors-β (“TGF-β”), vascular endothelial growth factor (“VEGF”), and epidermal growth factor (“EGF”).

5. A method of modifying a PRF centrifuge of the type having a chamber for containing the blood to form the PRF, the method comprising:
providing leads for bioelectrical stimulation of the chamber during a centrifugation process.

6. A centrifuge produced according to the method of claim 5.
